(19) United States
(12) Patent Application Publication
(10) Pub. No.: US 2026/0026718 A1
(43) Pub. Date: Jan. 29, 2026

(54) METHODS, SYSTEMS, AND COMPUTER READABLE MEDIA FOR MELANIN-CONCENTRATION-ADJUSTED PULSE OXIMETRY

(71) Applicant: The University of North Carolina at Chapel Hill, Chapel Hill, NC (US)

(72) Inventors: Arjun Putcha, Chapel Hill, NC (US); Kevin Schichlein, Wilmington, NC (US); Gaga Ellis, Brevard, NC (US); Bryson Wicker, Chapel Hill, NC (US); Wubin Bai, Chapel Hill, NC (US); Andrea Giovannucci, Sevilla (ES)

(21) Appl. No.: 18/998,008

(22) PCT Filed: Aug. 3, 2023
(86) PCT No.: PCT/US2023/029430

§ 371 Date: Jan. 23, 2025

Related U.S. Application Data
(60) Provisional application No. 63/394,941, filed on Aug. 3, 2022.

(51) Int. Cl. A61B 5/1455; A61B 5/00

(52) U.S. Cl. CPC A61B 5/14551; A61B 5/443

(57) ABSTRACT

Existing pulse oximeters have impaired accuracy in measuring blood oxygen levels (SpO2) in patients of color, where the presence of melanin in the skin tends to interfere with measurements of pulse oximetry, resulting in an overestimation of SpO2. Thus, the pulse oximeter often reports a higher oxygen saturation for patients with darker skin tones, often with increasing bias as the actual saturation (SaO2) decreases. This bias is further reinforced by calibration based on individuals with light skin pigmentation, which may lead to inequitable healthcare for patients of color. Our proposed solution is to modify current pulse oximetry calculations—which utilize the relative tissue absorbance of red and infrared light to estimate SpO2—to account for the concentration of melanin by additionally measuring the skin absorbance of UV˜A light. This derived concentration of melanin can then be used to modify the pulse oximetry algorithm output, thus estimating SpO2 more accurately for patients of color.

### PRIORITY CLAIM

This application claims the priority benefit of U.S. Provisional Patent Application Ser. No. 63/394,941 filed Aug. 3, 2022, the disclosure of which is incorporated herein by reference in its entirety.

### TECHNICAL FIELD

The subject matter described herein relates to pulse oximetry. More particularly, the subject matter described herein relates to pulse oximetry that is adjusted for differences in melanin concentrations of subjects.

### BACKGROUND

Pulse oximeters estimate blood oxygen saturation percentages in subjects (e.g., human subjects) using an optical sensor that transmits light into the subject's skin and measures the amount of light absorbed by the subject's blood. The amount of absorbed light is used to estimate the oxygen saturation percentage in the subject's arterial blood. One observed problem with conventional pulse oximeters in clinical settings is their tendency to overestimate oxygen saturation in patients of color. Our research suggests melanin—a key chromophore responsible for skin pigmentation—may be responsible for this overestimation of oxygen saturation which can in turn lead to diagnostic and therapeutic errors.

In light of these and other difficulties, there exists a need for improved methods, systems, and computer readable media melanin-concentration-adjusted pulse oximetry.

### SUMMARY

A pulse oximeter includes a first sensor for producing a first measurement indicative of a melanin concentration in a portion of the skin of a subject. The pulse oximeter further includes a second sensor for producing a second measurement indicative of light absorbed by melanin and arterial blood of the subject, which can be used to determine an initial modulation ratio, used in pulse oximetry to estimate blood oxygen levels. The pulse oximeter further includes a blood oxygen saturation percentage measurement generator for generating a third measurement based on the first and second measurements, wherein the third measurement is indicative of a percentage of oxygen saturation of arterial blood, corrected for the presence of melanin, of the subject.

In one example, the first sensor comprises at least one light source and at least one light detector configured to measure light absorbed by the melanin in the skin of the subject.

In one example, the at least one light source comprises at least one light emitting diode (LED) for emitting light in a wavelength range and the at least one first light detector comprises at least one photodiode configured to measure light of the wavelength range emitted by the at least one LED.

In one example, the wavelength range emitted by the at least one LED includes an ultraviolet to violet wavelength range.

In one example, the ultraviolet to violet wavelength range comprises about 390 nanometers (nm) to about 410 nm.

In one example, the first sensor utilizes at least one additional light source to improve sensitivity of melanin estimation.

In one example, the additional light source comprises of at least one light emitting diode (LED) for emitting light in a wavelength range, with at least one photodiode configured to uniquely measure the emitted wavelength.

In one example, the wavelength range emitted by the at least one LED includes a red wavelength (˜660 nm).

In one example, the wavelength range emitted by the at least one LED includes a near infrared wavelength (˜940 nm).

In one example, the second sensor comprises at least one light source and at least one light detector configured to measure light absorbed by the arterial blood of the subject.

In one example, the at least one light source comprises at least one first light emitting diode (LED) for emitting light in a wavelength range and the at least one first light detector comprises at least one photodiode configured to measure light of the wavelength range emitted by the at least one LED.

In one example, the wavelength range of the light emitted by the at least one LED includes at least one wavelength selected from a red wavelength range and at least one wavelength selected from an infrared wavelength range.

In one example, the at least one wavelength selected from the red wavelength range comprises 660 nanometers (nm) and the at least one wavelength selected from the infrared wavelength range comprises 940 nm.

In one example, the blood oxygen saturation percentage measurement generator is configured to utilize outputs from the first sensor to measure the melanin concentration.

In one example, the blood oxygen saturation percentage measurement generator is configured to utilize outputs from the second sensor to provide an initial estimate of the modulation ratio.

In one example, the blood oxygen saturation percentage measurement generator is configured to calculate an adjusted modulation ratio based on the first and second measurements and use the adjusted modulation ratio to generate the third measurement indicative of the percentage of oxygen saturation of the arterial blood of the subject.

In one example, the pulse oximeter includes signal enhancing optics for enhancing signals detected by the first and second sensors.

In one example, the signal enhancing optics include light blockers, optical filters, or polarizers.

In one example, the pulse oximeter includes post-acquisition signal enhancers for enhancing signals produced by the first and second sensors.

In one example, the post-acquisition signal enhancers implement wavelet transforms or machine learning algorithms to filter or smooth signals detected by the first and second sensors.

According to another aspect of the subject matter described herein, a method for melanin-concentration-adjusted pulse oximetry is provided. The method includes detecting, using a first sensor, a first measurement indicative of a melanin concentration in a portion of the skin of a subject. The method further includes detecting, using a second sensor, a second measurement indicative of light absorbed by melanin and arterial blood of the subject. The method further includes generating, based on the first and second measurements, a third measurement indicative of a percentage of oxygen saturation of arterial blood of the subject.

In one example, the first sensor comprises a melanin concentration sensor, the second sensor comprises an arterial blood light absorption sensor, and the blood oxygen saturation measurement adjusted is configured to calculate an adjusted modulation ratio using the first and second measurements.

In one example, generating the third measurement includes generating a blood oxygen saturation percentage measurement using the adjusted modulation ratio.

In one example, the first sensor includes at least one ultraviolet light source and the second sensor includes at least one red and at least one infrared light source.

In one example, the first sensor comprises a melanin concentration sensor, the second sensor comprises an arterial blood light absorption sensor, and generating the third measurement includes generating an adjusted modulation ratio based on the first and second measurements.

In one example, generating the third measurement includes mapping the adjusted modulation ratio to a blood oxygen saturation percentage value.

According to another aspect of the subject matter described herein, a non-transitory computer readable medium having stored thereon executable instructions that when executed by a processor of a computer control the computer to perform steps is provided. The steps include obtaining a first measurement generated by a first sensor and indicative of a melanin concentration in a portion of the skin of a subject. The steps further include obtaining a second measurement generated by a second sensor and indicative of light absorbed by melanin and arterial blood of the subject and from which an initial modulation ratio can be determined. The steps further include generating, based on the first and second measurements, a third measurement—an adjusted modulation ratio-indicative of a percentage of oxygen saturation of arterial blood of the subject.

The subject matter described herein can be implemented in software in combination with hardware and/or firmware. For example, the subject matter described herein can be implemented in software executed by a processor. In one exemplary implementation, the subject matter described herein can be implemented using a non-transitory computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

The subject matter described herein will now be explained with reference to the accompanying drawings, of which:

FIG. 1 is a graph of red/infrared modulation ratio (R) versus SaO₂ [18];

FIG. 2 is a graph illustrating optical absorbance spectra of blood components and melanin. HbR: deoxygenated hemoglobin; HbO2: oxygenated hemoglobin;

FIG. 3 illustrates images of synthetic melanin samples from greatest (0.15 mg/ml of water) to least (0 mg/ml of water);

FIG. 4 is a diagram illustrating penetration depth of light ranging from 150 to 1200 nm, image from the Wellman Center for Photomedicine;

FIG. 5 is a graph of absorption coefficient of melanin, deoxygenated hemoglobin (HbR), and oxygenated Hemoglobin (Hb), across 200-1000 nm wavelengths. Vertical lines indicate potential wavelength choices for UV, Red, and IR light respectively;

FIG. 6 is an image of an nRF development board (Left), melanin sensor (bottom right), optically analogous synthetic skin sample (top right);

FIG. 7 is a circuit diagram illustrating an exemplary design of a pulse oximeter capable of producing melanin-absorption-calibrated pulse oximetry measurements;

FIG. 8 is a graph illustrating relative absorbance of oxygenated blood and melanin sample vs deoxygenated blood;

FIG. 9 is block diagram illustrating a pulse oximeter capable of melanin-concentration-adjusted blood oxygen saturation percentage measurements;

FIG. 10 is a flow chart illustrating an exemplary process for blood melanin-concentration-adjusted blood oxygen saturation percentage measurements;

FIG. 11 is a graph of baseline modulation ratio (R_DC) vs melanin concentration (mg/mL);

FIG. 12 is a graph of SpO2 estimation vs melanin concentration (mg/mL);

FIG. 13 is a graph of calibrated absorption of 410 nm light versus melanin concentration;

FIG. 14 is a graph of the impact of the calibrated modulation ration on SpO2; and

FIG. 15 is a graph illustrating key features of support vector regression.

### DETAILED DESCRIPTION

Clinical Relevance-On Feb. 19, 2021, the Food and Drug Administration (FDA) issued the following statement: “Due to the accuracy limitations at the individual level, SpO2 provides more utility for trends over time instead of absolute thresholds.”[19]. The notion that pulse oximetry overestimates blood oxygen saturation—SpO2—in patients of color has been confirmed repeatedly across the years [4,5,6], leading the FDA to issue the above statement. However, it has also been shown that this overestimation increases as the true blood oxygen saturation decreases [5], meaning both the absolute value, and the trend in blood oxygen levels, are likely to appear less severe in patients of color, potentially resulting in improper treatment of patients of color. Since pulse oximetry can inform decisions for diagnostic and therapeutic interventions for patients of all ages [25,26,27], the significance of this problem cannot be considered minor.

### I. BACKGROUND

### Blood Oxygen

The oxygenation of tissue is a vital metric for the health of an individual, as oxygen is necessary for cellular respiration, specifically serving as the final electron acceptor in the mitochondrial electron transport chain [17]. The transfer of oxygen from lungs to tissue is accomplished primarily through the action of hemoglobin molecules, present within red blood cells. Generally, 97-98% of total oxygen content within blood is held by hemoglobin, with the remaining being dissolved within plasma. It is for this reason the approximation of blood oxygen levels—the amount of oxygen within the blood—through the measurement of oxygenated hemoglobin (HbO₂) and deoxygenated hemoglobin (Hb) is a reasonable assumption [14,15]

Generally, the oxygen saturation of arterial blood (SaO2) is defined as the ratio of HbO₂ concentration to total hemoglobin concentration

$${SaO}_{2} = [ {Hb O}_{2} ] / ( [ {Hb O}_{2} ] + [ Hb ] )$$

Since oxygen is only extracted by tissue from the capillaries, the relative concentrations of HbO₂ and Hb are the same throughout the arterial system. As such, the measurement of SaO2 can be done with an arterial blood gas test (ABG test), where a sample of blood is generally extracted from the radial or femoral arteries and analyzed to determine SaO₂ [12,14,15,16]. However, this system has a delayed response time for a single measurement of true oxygen saturation (SaO₂), limiting the potential for real-time monitoring. As such, pulse oximeters are used to noninvasively and continuously estimate oxygen saturation, denoted as SpO₂ [11,12]

### Pulse Oximeters

Because hemoglobin holds 97-98% of the total oxygen content within blood, the measurement of concentration ratios between Hb and HbO₂ provides a good approximation of blood oxygen levels. Since Hb and HbO₂ differentially absorb red and near-infrared (IR) light-typically, 660 and 940 nm wavelengths respectively—based on the Beer-Lambert law the relative absorbance of these two wavelengths can be used to estimate the blood oxygen level, SpO₂. Intuitively, as SaO₂ increases, the concentration of HbO₂ will increase while that of Hb decreases [11].

Additionally, both red and IR light allows for an optimized penetration of the light in tissue, allowing the absorbance of these wavelengths by blood to be conveniently measured at the finger tip. Absorbance (A) is a measure of how much light fails to pass through a medium, generally through absorbance by tissue. If T refers to the fractional transmission of light through a given medium, I refers to the intensity of light (input or received), and η refers to the efficiency with which emitted light reaches the tissue, and emerging light is captured by the photodetector, absorbance can be written as [18]:

$${A}_{overall} = - log ( {T}_{overall} ) = - log ( {I}_{out} / {I}_{in} )$$

$$Where :$$

$$log ( {T}_{overall} ) = log ( η ) + log ( {T}_{pigment} ) + log ( {T}_{skin} ) + log ( {T}_{bone} ) + log ( {T}_{venous} ) + log ( {T}_{arterial} ) + log ( {T}_{misc} )$$

For a given individual, generally the transmission through arterial blood dominates changes of Tₒᵥₑᵣₐₗₗ, mostly due to the increase in cross-sectional area of arteries in the systolic phase of the cardiac cycle. This allows us to hypothesize the following relationship between the change in absorbance over time [11,18]:

$$d ( {A}_{overall} ) / dt = - d log / dt ( {T}_{arterial} [ t ] )$$

Additionally, beer-lambert law correlates the absorbance A for a substance X at wavelength λ as following:

$${A}_{X}^{λ} = [ X ] {ε}^{λ} l ,$$

where [X] is the concentration of substance X, ελ is its molar extinction coefficient at wavelength λ, and I is the path length determined by the dimensional design between light source and photodetector. this is additive, meaning that for a mixture M with n components,

$${A}_{M}^{λ} = {\sum }_{i = 0}^{n} [ {X}_{i} ] {ε}_{i}^{λ} l$$

combining the above equations allows us to create the modulation ratio equation used to estimate SpO₂ [18]:

$$R = \frac{{dA}_{overall}^{660 nm}}{{dA}_{overall}^{940 nm}} \approx \frac{{A}_{AC}^{660 nm} / {A}_{DC}^{660 nm}}{{A}_{AC}^{940 nm} / {A}_{DC}^{940 nm}} = \frac{{( ( S {ε}_{{HbO}_{2}}^{660 nm} + ( 1 - S ) {ε}_{Hb}^{660 nm} ) \cdot Δ [ Arterial Hemoglobin ] \cdot l )}_{660 nm}}{{( ( S {ε}_{{HbO}_{2}}^{940 nm} + ( 1 - S ) {ε}_{Hb}^{940 nm} ) \cdot Δ [ Arterial Hemoglobin ] \cdot l )}_{940 nm}}$$

where S is the true SaO₂—written as a value between 0 and 1—and AC/DC refer to the amplitude and baseline absorbance, respectively, of a given wavelength. in practice, the absorbance of red (660 nm) and IR (940 nm) wavelengths are tracked over time, with the AC and DC (pulsatile and baseline) components of each extracted over time and used to calculate the R value. This R value is then mapped to the following experimentally derived curve to estimate SpO₂ [11, 18].

### II. INTRODUCTION

Sensing signals collected from traditional pulse oximeters are confounded by a number of physiological parameters, including peripheral vascular health and tissue composition. Our research indicates a dominating chromophore in skin—melanin—also contributes to the irregular absorption of light used in pulse oximetry. The modulation ratio R used in pulse oximetry is calculated as a function of the pulsatile and baseline components of light absorbance, and the only component thought to change is the absorbance of arterial blood. relying on the same curve to estimate SpO₂ from the R value assumes that the only component changing from patient to patient is the absorbance of arterial blood, as a result of varying concentrations of Hb and HbO₂. However, given that the total absorbance is also a function of skin pigmentation (melanin) as well, we theorize that patients of color could have a higher DC (baseline) absorbance at certain wavelengths, resulting in the ratio between AC and DC absorbance components being skewed.

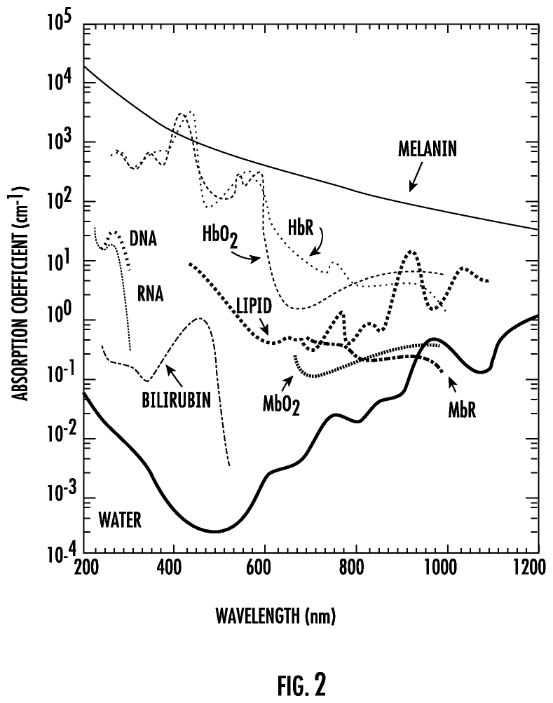

Referring to FIG. 2, it is apparent melanin also has a significant absorption coefficient, ε^{λ}ₘₑₗₐₙᵢₙ, at both red (660 nm) and IR (940 nm) wavelengths. As a result, the baseline absorbance of red and infrared light, A_{DC}^{660 nm} and A_{DC}^{940 nm}, will be higher, with A_{DC}^{660 nm} being higher due to the higher absorption coefficient of melanin at 660 nm. Because the two DC components do not increase similarly, the ratio of AC/DC absorbance components decreases more with red light than with infrared in the presence of melanin, resulting in the modulation ratio—R—becoming smaller. Since a smaller R value results in a higher estimate of SpO₂, theoretically the presence of melanin will result in an overestimation of SpO₂.

The above hypothesis has been observed in clinical settings repeatedly across the years [4,5,6], and has recently become a well-known public health concern due to the COVID-19 pandemic. On Feb. 19, 2021, due in part to a study by Sjoding et al. suggesting pulse oximeters overestimate SpO₂ in black patients [4,19], the FDA issued the following statement: “Due to accuracy limitations at the individual level, SpO₂ provides more utility for trends over time instead of absolute thresholds.” [19]. However, Bickler et al. has demonstrated the overestimation of SpO₂ in black patients increases as SaO₂ decreases, meaning the blood oxygen level trends in black patients may look less severe, due to increase overestimation of SpO₂, than in white patients [5]. Though the sample size in the study was relatively small, with only 11 darkly pigmented individuals and 10 lightly pigmented individuals, the math behind oximetry calculations, shown in the Methods section, suggests this may be a valid concern. And given the importance of pulse oximetry in monitoring of Intensive Care Units (ICUs) [1], as well as its potential to reduce mortality rates in many groups, including children [10], this observation warrants further investigation and solutions. It should also be noted the impact of the overestimation of SpO₂ may not have obvious impact—instead, it may subconsciously influence healthcare providers' decisions, thus contributing to the inequities prevalent in many healthcare systems.

In pre-hospital care, it has been shown that using pulse oximetry to titrate low-dose oxygen therapy (generally targeting an SpO₂ between 88 and 92%) can reduce mortality in chronic obstructive pulmonary disease (COPD) [25]. Castillo et al. have shown to a reasonable degree of confidence neonatal SpO₂ levels between 85-93% are associated with a generally considered safe level of blood oxygen content (where above and below this range can prove dangerous) [26]. Pulse oximetry has also been found to improve detection of congenital heart defects in neonates. Specifically, Ewer et al. found that SpO₂ readings less than 95% in either an infant's right arm or foot (or a difference of more than 2% between limbs) triggered referrals for echocardiograms, in turn increasing the detection of congenital heart defects compared with antenatal ultrasonography and clinical examination alone [27]. Given that respiratory therapies for patients of many ages rely upon titrating patient SpO₂ levels to a specific level, or identifying cutoff points for diagnostic and therapeutic interventions, the fact that SpO₂ readings may be especially inaccurate in patients of color may be of great concern for clinicians.

As such, in the following we propose a method to account for the presence of melanin in patients in order to correct the modulation ratio to better estimate SpO₂ in patients of color. Specifically, we intend to show the measurement of violet light absorbance can be used to accurately and sensitively measure a range of melanin concentrations, thus allowing us to prevent overcorrection of the modulation ratio. Additionally, we will present the math model we intend to use to tune the modulation ratio, R, to correct for the presence of melanin.

### III. METHODS

### Synthetic Skin

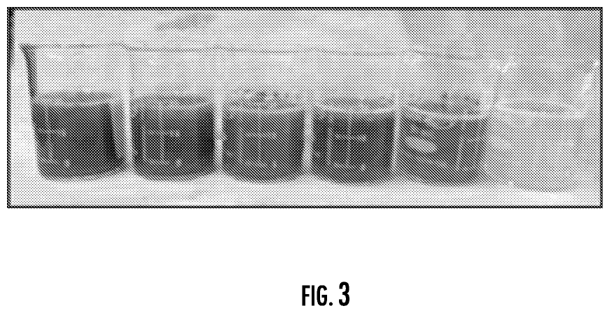

In order to determine the melanin concentrations needed to test the UV sensor in synthetic skin samples, we began with the data presented in Smit et al [21]. Their creation of liquid skin phantoms over a range of eumelanin concentrations representing Fitzpatrick skin types I through VI was of particular interest as many studies focused solely on the lighter skin types I and II. We took Smit et al's highest melanin concentration representing Fitzpatrick skin type VI and used a 25% increase in that concentration to generate our maximum concentration to test. Furthermore, we resolved to use a sample lacking melanin to test as a minimum concentration. The resulting melanin concentration range used for testing was zero to. 15 mg/mL as shown in FIG. 3 and table 1.

To mimic the optical properties of skin, we created two layers of synthetic skin. The first layer (synthetic tissue) was a mixture of horse blood, agar, and distilled water. For every 97 mL of water, 3 grams of agar and 1 mL of horse blood was added. All components were manually stirred in to obtain a roughly homogenous solution.

Since it is possible the oxygenation status of the blood can influence the absorption of UV-A light, we tested the relative absorption of two forms of horse blood: one 5 mL sample mixed with 10.818 μL of hydrogen peroxide, and the other with 12.5 mg of sodium dithionide. The former serves to oxygenate the horse blood, while the latter de-oxygenates. The chosen concentration of hydrogen peroxide and sodium dithionide were derived from White et al. and Briley-Sæbø et al. respectively [24, 23]. As we will show in the Results section, the difference in UV-A absorbance between the two samples was negligible compared to the absorbance of melanin, allowing us to neglect the impact of blood oxygen saturation in the creation of the synthetic skin. As such, the ‘tissue’ layer of the synthetic skin needed only horse blood, agar, and distilled water.

The second layer (synthetic melanin) was a mixture of synthetic melanin, obtained from sigma aldrich (M8631), distilled water, and agar. Since melanin is more difficult to dissolve in neutral pH solutions, first we added the correct dosage of melanin (described above) to the distilled water, and placed it on a hotplate stirrer set to 90 degrees Celsius at 5500 RPM for 2 hours. After this, we added the required amount of agar (3 grams for every 97 mL of water), and manually stirred. We repeated this six times, with the concentration of melanin starting at 0 mg/ml of water and increasing by increments of ˜0.03 mg/mL. FIG. 3 shows the synthetic melanin samples at each concentration while table 1 shows the exact concentration values that were achieved.

TABLE 1
Synthetic melanin samples I through
VI and their Concentration values

| Sample | Concentration (mg/mL of water) |
|---|---|
| I | 0 |
| II | 0.0360 |
| III | 0.0566 |
| IV | 0.0992 |
| V | 0.116 |
| VI | 0.150 |

To set both layers of synthetic skin, we microwaved the solutions in the microwave in small increments—8 to 15 seconds—at a low power level until the desired viscosity was achieved. Once done, we let the solution sit at room temperature, covered with aluminum foil to prevent excessive dehydration of the sample.

The assembly of the skin sample was done by cutting a 1 cm thick sample of the synthetic tissue sample, and placing a 1.2 mm thick sample of the synthetic melanin sample on top of it. Light was shone through both tissue samples simultaneously.

### Mathematics Model

As mentioned earlier, SpO2 estimation relies upon the ratio of pulsatile and baseline absorption of red and infrared light over time. The modulation ratio used to estimate SpO₂, R, follows the following equation:

$$R = \frac{{A}_{AC}^{660 nm} / {A}_{DC}^{660 nm}}{{A}_{AC}^{940 nm} / {A}_{DC}^{940 nm}} = {R}_{AC} \frac{{A}_{DC}^{940 nm}}{{A}_{DC}^{660 nm}} = {R}_{AC} {R}_{DC}$$

A key assumption made in the conversion of the modulation ratio to SpO₂ is that the main physiological feature that changes from patient to patient is the oxygen saturation of blood. Of course, it is known sensing signals collected from traditional pulse oximeters are confounded by a number of physiological features, such as peripheral vascular flow and vascular elasticity, and many efforts are made to account for such variance. However, while a number of past papers purport a racial bias in pulse oximetry, to our knowledge no past papers have provided a substantiated hypothesis for how race—a sociological structure—affects the estimation of SpO₂. Similarly, to our knowledge, while some papers conclude race plays no significant confounding role in SpO₂ estimation, no justification has been proposed for why this may hold true.

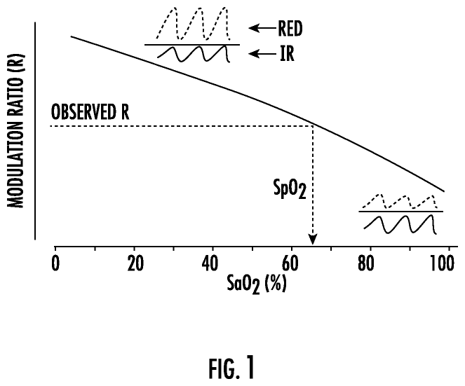

We propose a theory that melanin—a key chromophore found in the epidermis, and is present in different concentrations across human populations—may be responsible for the clinical observation of the overestimation of SpO₂ in patients of color. Looking at FIG. 1b, it is apparent melanin also has a significant absorption coefficient, melanin, at both red (660 nm) and IR (940 nm) wavelengths. As a result, we anticipate the baseline absorbance ratio (R_{DC}) of red and infrared light will be higher; Additionally, we suspect the resulting additional absorption of red light, A_{DC}^{500 nm}, will be higher due to the higher absorption coefficient of melanin at 660 nm. Baseline absorption ratio, R_{DC}, can be understood as the following expression:

$${R}_{DC} = \frac{{A}_{DC}^{940 nm}}{{A}_{DC}^{660 nm}} = \frac{[ melanin ] {ε}_{melanin}^{940 nm} {l}_{melanin}^{940 nm} + {{\sum }_{i = 1}^{n} [ X ]}_{i} {ε}_{i}^{940 nm} {l}_{i}^{940 nm}}{[ melanin ] {ε}_{melanin}^{660 nm} {l}_{melanin}^{660 nm} + {{\sum }_{i = 1}^{n} [ X ]}_{i} {ε}_{i}^{660 nm} {l}_{i}^{660 nm}}$$

Where

$${{\sum }_{i = 1}^{n} [ X ]}_{i} {ε}_{i}^{λ nm} {l}_{i}$$

refers to the cumulative absorption—determined by the concentration [X] of the given substance X, absorptivity at a given wavelength λ,

$${ε}_{i}^{λ nm} ,$$

and the path length light travels through a substance X—of all n substances in the tissue at a given wavelength λ save for the contribution of melanin. The contribution of melanin to the absorbance of light is explicitly defined in the first term in the numerator and denominator,

$$[ melanin ] {ε}_{melanin}^{λ nm} {l}_{melanin} .$$

Because the only variable that changes between red and infrared baseline light absorption is the absorptivity, ε, and because the absorption of infrared light

$$( {ε}_{melanin}^{940 nm} )$$

is less than that of red light

$$( {ε}_{melanin}^{660 nm} )$$

the R_{DC} value will consistently be multiplied by a fraction less than 1. And since we assume the pulsatile component of absorption, R_{AC}, is unaffected by the presence of melanin, this reduction in R_{DC} will in turn artificially inflate the measured SpO₂.

From a mathematical perspective, we know

$$R = \frac{{A}_{AC}^{660 nm} / {A}_{DC}^{660 nm}}{{A}_{AC}^{940 nm} / {A}_{DC}^{940 nm}} = {R}_{AC} \frac{{A}_{DC}^{940 nm}}{{A}_{DC}^{660 nm}} = {R}_{AC} {R}_{DC} ,$$

and we can also reasonably assume the presence of melanin primarily affects the baseline component of absorption, R_{DC}, as the concentration, absorptivity, and path length of melanin does not significantly change over time. As such, a reduction in R_{DC} will result in a lower R value. Given the higher absorbance of 660 nm light by melanin than 940 nm, we can understand the baseline component of absorption to be:

$${R}_{DC} = \frac{{A}_{DC}^{940 nm}}{{A}_{DC}^{660 nm}} = \frac{\begin{matrix}( Δ [ melanin ] + {[ melanin ]}_{assumed} ) \\ {ε}_{melanin}^{940 nm} {l}_{melanin}^{940 nm} + {{\sum }_{i = 1}^{n} [ X ]}_{i} {ε}_{i}^{940 nm} {l}_{i}^{940 nm}\end{matrix}}{\begin{matrix}( Δ [ melanin ] + {[ melanin ]}_{assumed} ) \\ {ε}_{melanin}^{660 nm} {l}_{melanin}^{660 nm} + {{\sum }_{i = 1}^{n} [ X ]}_{i} {ε}_{i}^{660 nm} {l}_{i}^{660 nm}\end{matrix}}$$

From this, we can create a new variable, R_{DC}_{_{new}}, to alter R_{DC} by accounting for variable changes in melanin concentration.

$${R}_{{DC}_{new}} = \frac{{A}_{DC}^{940 nm} - Δ [ melanin ] {ε}_{melanin}^{940 nm} {l}_{melanin}^{940 nm}}{{A}_{DC}^{660 nm} - Δ [ melanin ] {ε}_{melanin}^{660 nm} {l}_{melanin}^{660 nm}}$$

Finally, we have that

$${R}_{new} = {R}_{AC} {R}_{{DC}_{new}} = \frac{{A}_{AC}^{660 nm} [ {A}_{DC}^{940 nm} - Δ [ melanin ] {ε}_{melanin}^{940 nm} {l}_{melanin}^{940 nm} ]}{{A}_{AC}^{940 nm} [ {A}_{DC}^{660 nm} - Δ [ melanin ] {ε}_{melanin}^{660 nm} {l}_{melanin}^{660 nm} ]}$$

Since all values, save for the concentration of melanin (Δ[melanin]), can be found with current pulse oximeters, the only modification required from a hardware perspective is the inclusion of a method to estimate melanin concentration. It is for this reason our efforts are focused on developing reliable methods to estimate melanin concentration.

### Wavelength Selection for Melanin Sensor

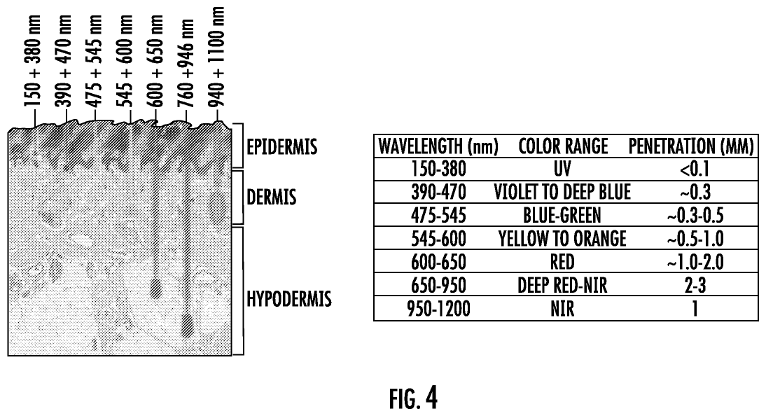

There are two primary considerations in selecting the ideal wavelength to measure melanin concentration. First, the penetration depth of a wavelength affects our understanding of light absorbance. FIG. 4, from the Wellman Center for Photomedicine, depicts the penetration depth of light ranging from 150 to 1100 nm.

Because our device uses reflectance spectrometry—meaning it measures the amount of light reflected from the tissue, not transmitted through—a greater penetration depth results in a greater chance for deeper tissue to absorb light. Since melanin is located in the basal layer of the epidermis, a shallower penetration depth is ideal to ensure the measured absorbance of light is affected primarily by absorbance of light from melanocytes, not the tissue below. For this reason, our ideal wavelength is shorter in wavelength, likely 500 nm or less.

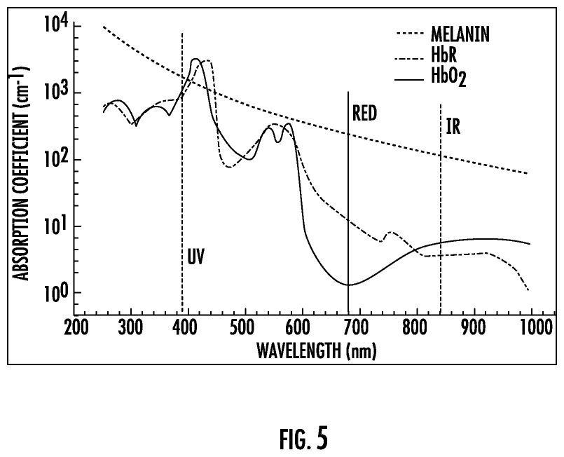

Our second consideration is whether the oxygenation status of an individual can significantly affect the absorbance of our chosen wavelength. From FIG. 5, it's apparent the UV light (UV-A, UV-B, and UV-C) all have certain wavelength ranges where the differential absorbance of light between oxygenated and deoxygenated hemoglobin is roughly equivalent. However, UV-B and UV-C light are especially dangerous for humans, due in part to the greater amount of energy contained [22]. As such, the range of 380-420 nm is of particular interest for the measurement of melanin.

To confirm the ideal wavelength to use, we used a spectrometer to measure the absorbance of a 1-cm diameter 4-mL cylinder of the oxygenated and deoxygenated blood, as well as a 1.2 mm thick sample of synthetic melanated skin, at the second lowest concentration we could test: 6 mg/ml of water. Our results from this experiment are shown in the Results section. LED-Photodiode Selection

Because our wavelength selection experiment suggested the ideal wavelength to measure melanin is between 390 to 410 nm, we selected four LEDs, each with different wavelengths and intensities of emission. Table 2 shows the properties of the four LEDs we tested.

| Name | Wavelength | Current |
|---|---|---|
| SM0603UV-405 | 405 nm | 25 mA |
| SM0603UV-395 | 395 nm | 25 mA |
| CS63CUV365C | 362 nm | 30 mA |
| SM0603UV-410 | 410 nm | 25 mA |

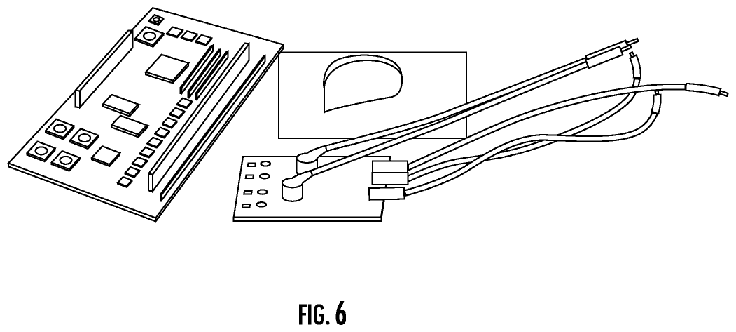

When searching for photodiodes, we found one particular photodiode, GVBL-S12SD, had a much higher responsivity compared to all others on the market (0.68 A/W at 405 nm, compared to 0.18 A/W for most alternatives). As such, we designed a preliminary melanin sensor to test the sensitivity and reliability of the four combinations of LEDs and photodiodes (PDs). FIG. 6 shows the primary components for our testing setup: an nRF development board for flashing firmware onto the device, our melanin sensor, and an optically analogous skin sample (protocol described in the Methods section).

To determine the ideal LED-PD combination, we measured the readings on the photodiode for when each LED was turned on, for all six samples of synthetic skin (each at increasing concentrations of melanin). The ideal LED-PD combination would be the one with the greatest difference in absorbance measurement between each concentration of melanin, and the most consistent readings. Our results for this experiment will be shown in the Results section, we are currently in the process of running this experiment.

### Pulse Oximeter Design

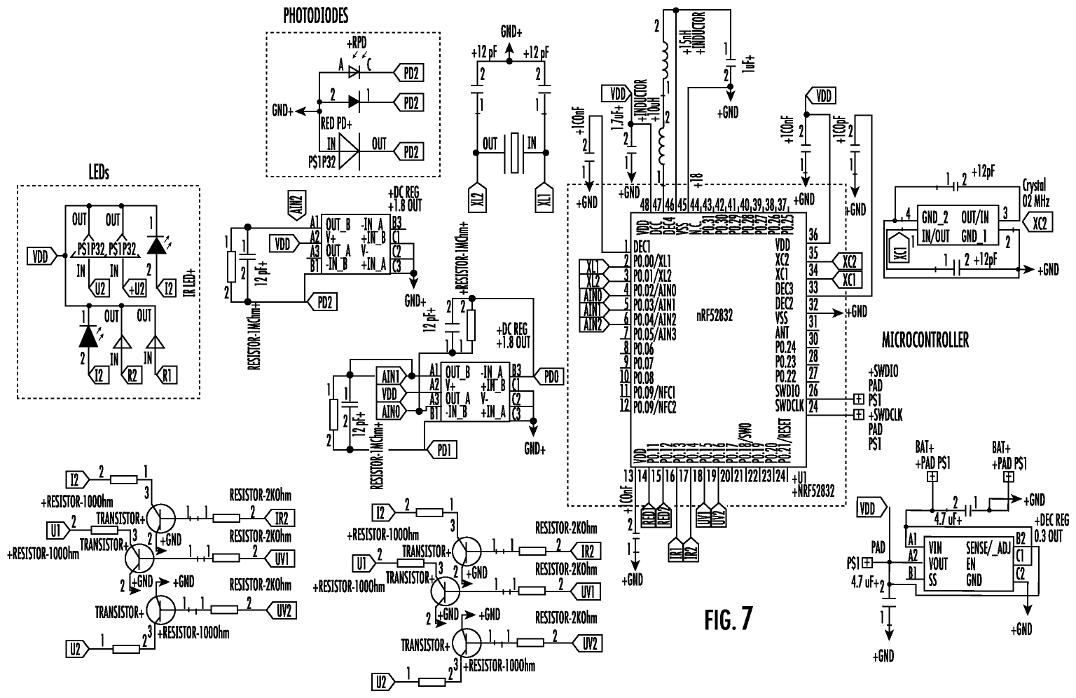

In addition to the consideration for wavelength choice to measure melanin, our pulse oximeter is designed to incorporate three distinct photodiodes, each uniquely selective for red, infrared, or UV-A light. This allows us to place each sensor relatively close to one another without a high chance for excessive cross talk between the photodiodes. FIG. 7 depicts the schematic for our pulse oximeter design.

There are six LEDs, two for each wavelength (red, IR, and UV-A), and three photodiodes, one for each wavelength. The presence of two LEDs for every PD allows us to maximize light emission and minimize thermal damage over time, thus improving our Signal-to-Noise Ratio (SNR).

### IV. RESULTS

### Wavelength Selection for Melanin Concentration Estimation

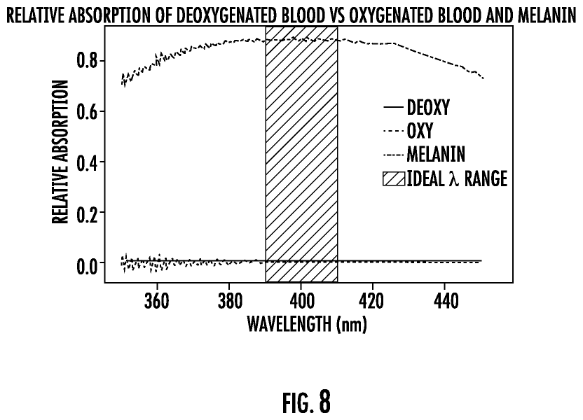

Using a spectrometer calibrated to the absorbance of a 1-cm diameter, 4 mL empty vial, we measured the absorbance of three samples: one with oxygenated blood and one with deoxygenated blood (created as described in the Synthetic Skin section), and one with a 1.2 mm thick sample of the third lowest concentration of melanin (0.0566 mg/ml of water) in our sample range placed within. A light is shown through the glass vial, and a spectrometer absorbs light from the other side. FIG. 8 shows the relative absorbance of the oxygenated blood sample and melanin compared to the deoxygenated blood sample.

From FIG. 8, it is clear that across all wavelengths between 350 and 450 nm, the oxygenation status of blood doesn't significantly affect the absorbance of light. Additionally, it is also apparent the wavelengths between 390 and 410 nm may be ideal to ensure differences in absorbance of light are due to changes in melanin concentration.

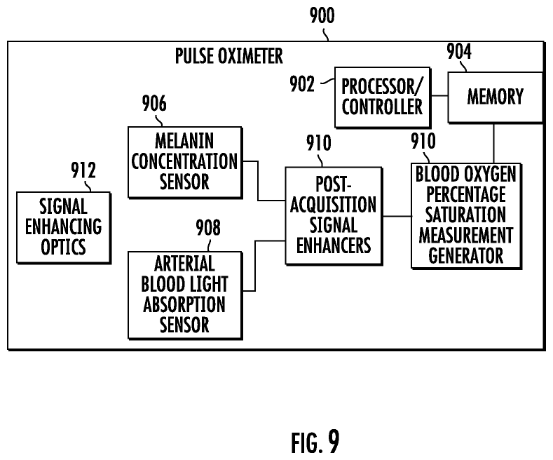

FIG. 9 is a block diagram illustrating an exemplary overall design for a pulse oximeter with improved accuracy for subjects with different skin tones. In FIG. 9, pulse oximeter 900 includes at least one processor/controller 902 and a memory 904. Pulse oximeter 900 further includes a melanin concentration sensor 906 and an arterial blood light absorption sensor 908. A blood oxygen saturation percentage measurement generator 910 receives measurements from melanin concentration sensor 906 and arterial blood light absorption sensor 908 and produces a blood oxygen saturation percentage measurement that is adjusted based on the melanin concentration measured by melanin concentration sensor 906.

In one example, melanin concentration sensor 906 includes at least one light source and at least one light detector configured to measure light absorbed by the melanin in the skin of the subject. The light source may include one or more LEDs, and the light detector may include one or more photodiodes. The light emitted by the LED(s) may include at least one wavelength in an ultraviolet range, for example, a range of from about 390 nm to about 410 nm.

In one example, arterial blood light absorption sensor 908 includes at least one light source and at least one light detector configured to measure light absorbed by the oxygenated arterial blood of the subject. The light source may one or more LEDs, and the light detector may include one or more photodiodes. The light emitted by the LED may include at least one wavelength in a red (wavelength=660 nm) or infrared (wavelength=940 nm) wavelength range.

According to another aspect of the subject matter described herein, multi-channel melanin sensing can be used to obtain more accurate melanin concentration measurements. For example, sensor 908 may be used in combination with sensor 906 to measure melanin concentration. Using red and infrared wavelength light in addition to ultraviolet light to measure melanin concentration may improve the sensitivity of melanin concentration measurements. In addition, because red and infrared wavelength light is already used in pulse oximeters to measure blood oxygen saturation, additional LEDs and photodiodes are not needed to enhance the melanin concentration measurement, resulting in a modular pulse oximeter design.

Blood oxygen saturation percentage measurement generator 910 may, in one example, calculate an adjusted modulation ratio based on the melanin concentration measurement and use the adjusted modulation ratio to generate the measurement indicative of the percentage of oxygen saturation of the arterial blood of the subject. For example, blood oxygen saturation percentage measurement generator 910 may compute the modulation ratios R_{DC}_{_{NEW}} and R_{NEW} using the equations described above and then use stored mappings between the modulation ratio and blood oxygen saturation percentage to generate a patient-melanin-concentration-adjusted blood oxygen saturation percentage measurement. In one example, blood oxygen saturation percentage measurement generator 910 may be implemented using a neural network, such as a recurrent neural network, or a regression model, such as Support Vector or Multiple Linear Regression, trained based on measurements of blood oxygen saturation percentage from a different source than sensor 908. One example of measurements that can be used for training the neural network includes arterial blood gas measurements.

Pulse oximeter 900 may also include signal enhancing optics 912 and post-acquisition signal enhancers 914 to further enhance the accuracy of blood oxygen saturation measurements output by pulse oximeter 900. In one example, signal enhancing optics 912 may include light blockers positioned around or near the photodiodes to block extraneous light and improve the signal to noise ratio. In another example, signal enhancing optics 912 may include polarizers placed optically in front of the LEDs to improve the penetration depth of red and infrared light, allowing for better reading of light absorption from the blood flowing through the radial artery. The polarizers may be implemented as a film or integrated within housing material of each LED. Post-acquisition signal enhancers 914 may implement wavelet transforms to filter/smooth the detected signals or machine learning algorithms (such as recurrent neural networks) to simultaneously filter, smooth, and calculate blood oxygen saturation percentage.

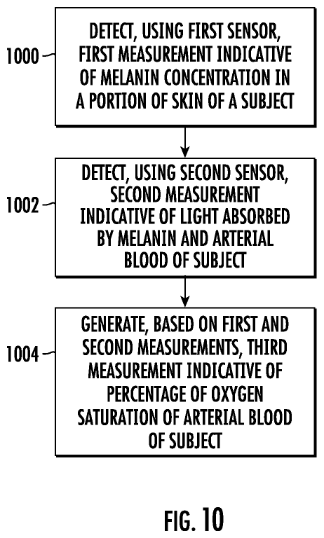

FIG. 10 is a flow chart illustrating an exemplary process for melanin-concentration-adjusted pulse oximetry. Referring to FIG. 10, in step 1000, the process includes detecting, using a first sensor, a first measurement indicative of a melanin concentration in a portion of the skin of a subject. For example, melanin concentration sensor 906 may generate red and/or infrared light that is used to illuminate a portion of a subject's skin, and one or more photodetectors may receive transmitted or reflected light and generate a signal indicative of the amount of light absorbed by melanin in the subject's skin. The amount of light absorbed by the melanin may be mapped to a melanin concentration value, for example, using mapping stored in memory 904.

In step 1002, the process includes detecting, using a second sensor, a second measurement indicative of light absorbed by melanin and arterial blood of the subject. For example, arterial blood light absorption sensor 908 may generate ultraviolet light that is used to illuminate, through the subject's skin, blood flowing in an artery of the subject. One or more photodetectors may detect a portion of the ultraviolet light transmitted through or reflected by the arterial blood and generate an output signal indicative of oxygen saturation percentage of the subject's arterial blood but also of melanin concentration in the subject's skin.

In step 1004, the process includes generating, based on the first and second measurements, a third measurement indicative of a percentage of oxygen saturation of arterial blood of the subject. For example, blood oxygen saturation percentage measurement generator 910 may receive output generated by melanin concentration sensor 906 and arterial blood light absorption sensor 908, calculate and adjusted modulation ratio using the equations described herein, and map the adjusted modulation ratio to a blood oxygen saturation percentage value using stored mappings between modulation ratios and blood oxygen saturation percentages. In one example, the stored mappings may be similar to those illustrated in FIG. 1, except that the modulation ratio used to make the mappings will be R_{new} instead of R.

The following section presents an updated mathematical model for pulse oximeter calibration and experimental and theoretical bases for the updated mode.

### Theoretical Basis for Impact of Melanin

Sensing signals collected from traditional pulse oximeters are confounded by a number of skin chromophores, among which the most dominating one is skin pigmentation (dictated by melanin concentration). Additionally, the modulation ratio R is calculated as a function of the pulsatile and baseline components of light absorbance, and the only component thought to change is the absorbance of arterial blood. Relying on the same curve to estimate SpO₂ from the R value assumes that the only component changing from patient to patient is the absorbance of arterial blood, as a result of varying concentrations of Hb and HbO₂. However, given that the total absorbance is also a function of skin pigmentation (melanin) as well, patients of color could have a higher DC (baseline) absorbance at certain wavelengths, resulting in the ratio between AC and DC absorbance components being skewed.

From FIG. 2, it is apparent melanin also has a significant absorption coefficient, melanin, at both red (660 nm) and IR (940 nm) wavelengths. As a result, the baseline absorbance of red and infrared light,

$${A}_{DC}^{660 nm} and {A}_{DC}^{940 nm} ,$$

will be higher, with

$${A}_{DC}^{660 nm}$$

being higher due to the higher absorption coefficient of melanin at 660 nm. Because the two DC components do not increase similarly, the ratio of AC/DC absorbance components decreases more with red light than with infrared in the presence of melanin, resulting in the modulation ratio—R—becoming smaller. Since a smaller R value results in a higher estimate of SpO2, theoretically the presence of melanin will result in an overestimation of SpO2.

From a mathematical perspective, we know

$$R = \frac{{A}_{AC}^{660 nm} / {A}_{DC}^{660 nm}}{{A}_{AC}^{940 nm} / {A}_{DC}^{940 nm}} = {R}_{AC} \frac{{A}_{DC}^{940 nm}}{{A}_{DC}^{660 nm}} = {R}_{AC} {R}_{DC} ,$$

and we can also reasonably assume the presence of melanin primarily affects the baseline component of absorption, R_{DC}, as the concentration, absorptivity, and path length of melanin does not significantly change over time. As such, a reduction in R_{DC} will result in a lower R value. Given the higher absorbance of 660 nm light by melanin than 940 nm, we can understand the baseline component of absorption to be:

$${R}_{DC} = \frac{{A}_{DC}^{940 nm}}{{A}_{DC}^{660 nm}} = \frac{( Δ [ melanin ] + {[ melanin ]}_{assumed} ) {ε}_{melanin}^{940 nm} {l}_{melanin} + {{\sum }_{i = 1}^{n} [ X ]}_{i} {ε}_{i}^{940 nm} {l}_{i}}{( Δ [ melanin ] + {[ melanin ]}_{assumed} ) {ε}_{melanin}^{660 nm} {l}_{melanin} + {{\sum }_{i = 1}^{n} [ X ]}_{i} {ε}_{i}^{660 nm} {l}_{i}}$$

Because the only variable that changes between red and infrared baseline light absorption is the absorptivity, ε, and because the absorption of infrared light

$$( {ε}_{melanin}^{940 nm} )$$

is less than that of red light

$$( {ε}_{melanin}^{660 nm} )$$

due to melanin, the R_{DC} value will consistently be multiplied by a fraction less than 1, thereby artificially inflating the measured SpO₂.

### Experimental Basis for Impact of Melanin

To experimentally determine the effects of melanin on SpO₂ estimation, we made use of the Ocean XR2 Spectrometer to measure the transmission of red and infrared light through samples of melanated tissue (composed of water, blood, and dissolved melanin). We created 5 samples, starting at 0.03 mg/mL of melanin, and going up to 0.15 mg/mL, thus representing the reasonable range of human epidermal melanin concentration. For reference, we also measured the transmission of light through a sample containing water and blood, but no melanin. Additionally, for each class of samples, we made 10 identical samples and took an average of light transmission for each class.

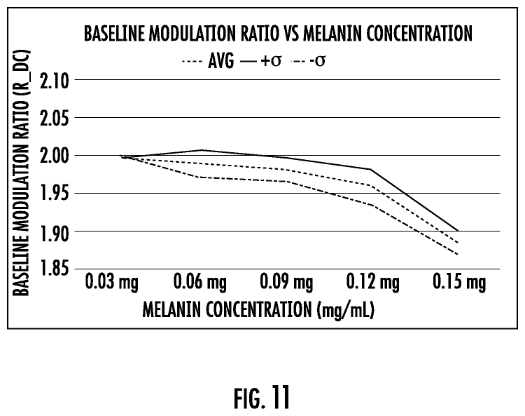

We then measured the spectral absorbance of light passing through each sample—1 centimeter in path length. We calibrated the absorbance of light passing through the 5 melanated samples with that of the sample containing no melanin. This allowed us to extract the additional contribution of melanin to light absorption. We then calculated the ratio of absorbance of infrared and red light for each calibrated sample. Finally, we converted this ratio of transmission to a ratio of absorbance—as is used in pulse oximetry calculation—and plotted the results in FIG. 11. The conversion of light transmission percentage (% T) to light absorbance is accomplished by the following equation: A=2−log (% T).

As is apparent from FIG. 11, there is a marked reduction in the baseline modulation ratio, R_{DC}, with respect to melanin concentration. Additionally, if we assume the ratio of pulsatile absorption of red and infrared light, R_{AC}, is constant regardless of the presence of melanin, we find the impact of the change in baseline modulation ratio due to melanin concentration will increase the estimated blood oxygen level, SpO₂.

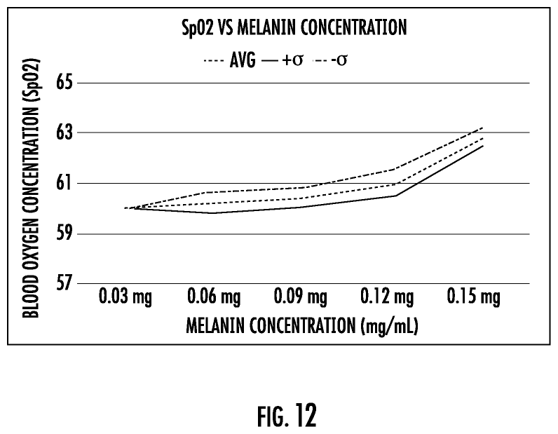

In industry, a standard formula to convert the Modulation Ratio, R, to SpO₂ is 110-25·R. If we let R=R_{AC}R_{DC}, and let R_{AC} equal 1 for simplicity's sake, we find a significant increase in SpO₂ estimation as shown in FIG. 12.

Given that the same concentration and type of blood was used for all samples, the observed increase in SpO₂ estimation suggests that the contribution of melanin confirms clinical observations of overestimation in SpO₂.

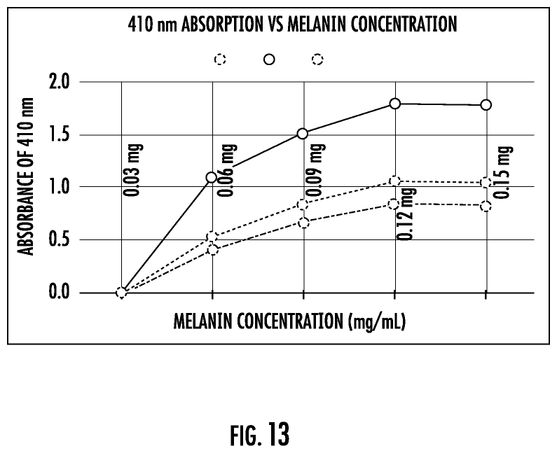

Using the same conversion methodology proposed above, we also measured the absorbance of 410 nm light due to increasing concentrations of melanin, and derived the results shown in FIG. 13.

There are a number of other wavelengths that exhibit significant changes in absorption due to melanin concentration, the above selection simply demonstrates one such example. It should also be noted, a 0.1 change in absorption corresponds to a 180-mV change in photodiode output from our device. Additionally, the global noise affecting our third wavelength channels ranges between 4-10 mV, making our observed increase in absorption as measured by our device statistically significant compared to our global noise. The above results support the notion that melanin contributes to the overestimation of SpO₂, and the use of a third wavelength can be effectively utilized to differentiate different melanin concentrations.

Mathematical Model behind Melanin-based SpO₂ Calibration

To correlate our device's absorbance values to that of the commercial spectrometer device, we can use the following regression model:

$$[ M ] = {α [ X ]}_{λ} + {β [ X ]}_{660} + {γ [ X ]}_{940} + δ$$

Where [M] represents the epidermal melanin concentration derived from the commercial spectrometer device; α, β, and γ are experimentally derived constants that weight the importance of each wavelength's absorption in the same tissue, X, and δ is a constant representing the constant displacement of values from the origin. To solve this regression model, we can use Support Vector Regression, Multiple Linear Regression, or a number of other models. For details on the mathematical implementation of SVR, please see the Mathematical Model: SVR section. The key advantage SVR provides us is the ability to use the kernel trick to transform a nonlinear input—as may be the case with the association of light absorbance and melanin concentration—into a linearly separable space.

$$Additionally , Because we know {R}_{{DC}_{new}} = \frac{{A}_{DC}^{940 nm} - Δ [ melanin ] {ε}_{melanin}^{940 nm} {l}_{melanin}^{940 nm} ]}{{A}_{DC}^{660 nm} - Δ [ melanin ] {ε}_{melanin}^{660 nm} {l}_{melanin}^{660 nm} ]} , we$$

$$can also say$$

$${R}_{{DC}_{new}} = \frac{{A}_{DC}^{940 nm} ( 1 - \frac{Δ [ melanin ] {ε}_{melanin}^{940 nm} {l}_{melanin}^{940 nm}}{{A}_{DC}^{940 nm}} )}{{A}_{DC}^{660 nm} ( 1 - \frac{Δ [ melanin ] {ε}_{melanin}^{660 nm} {l}_{melanin}^{660 nm}}{{A}_{DC}^{660 nm}} )} = {R}_{DC} = \frac{( 1 - \frac{Δ [ melanin ] {ε}_{melanin}^{940 nm} {l}_{melanin}^{940 nm}}{{A}_{DC}^{940 nm}} )}{( 1 - \frac{Δ [ melanin ] {ε}_{melanin}^{660 nm} {l}_{melanin}^{660 nm}}{{A}_{DC}^{660 nm}} )} = {R}_{DC} \cdot [ M ] ,$$

where [M] is the same adjustment factor derived from our SVR model that allows us to adjust our modulation ratio R across a wide range of SpO₂ values. Because of this, the problem becomes simply

$${R}_{new} = {R}_{AC} {R}_{{DC}_{new}} = {R}_{AC} {R}_{DC} \cdot [ M ]$$

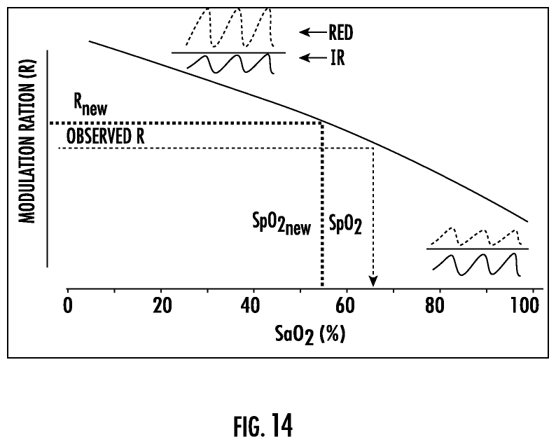

The above methodology allows us to transform the absorbance of three wavelengths into a tuning factor [M] used to adjust R_{DC}, and in turn the modulation ratio R_{new}. FIG. 14 depicts the impact R_{new} has on SpO₂ estimation. Since the tuning factor [M] will counter the reduction in R value due to the presence of melanin by inflating the R value, the estimation of SpO₂ will in turn decrease, thus mitigating the overestimation of blood oxygen saturation in patients of color.

### Mathematical Model: Support Vector Regression

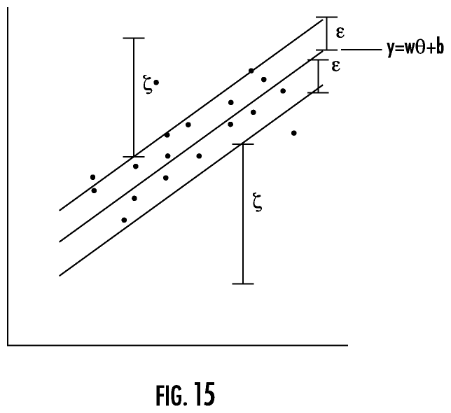

Referring to FIG. 15, we see a standard regression problem where we attempt to fit n points to the form y=wθ+b, where y is the output variable, w is a vector of parameter constants, θ a vector of input variables, and b is a y intercept. In our case, we can consider the output variable, y, to be the epidermal melanin concentration, [M]. Additionally, w is a row vector [α β γ], and θ is a column vector

$$[ \begin{matrix}λ \\ 660 \\ 940\end{matrix} ]$$

of the absorbances at three wavelengths. This gives us the following equation:

$$[ M ] = [ \begin{matrix}α & β & γ\end{matrix} ] [ \begin{matrix}λ \\ 660 \\ 940\end{matrix} ] + δ$$

Because our input data may not be linearly related to our output variable, we make use of the kernel trick to map our nonlinear input into a linearly separable space. To do so, we satisfy the following problem:

$$f ( θ , y ) = {max}_{α} \{ {\sum }_{i = 1}^{n} {α}_{i} - \frac{1}{2} {\sum }_{i = 1}^{n} {\sum }_{j = 1}^{n} [ {α}_{i} {α}_{j} {y}_{i} {y}_{j} \cdot K ( {θ}_{i}^{T} {θ}_{j}^{T} ) ] \}$$

$$s . t . \{ \begin{matrix}0 \leq {α}_{i} \leq C for all i \\ {\sum }_{i = 1}^{n} {α}_{i} {y}_{i} \\ K is a Gaussian RBF Kernel\end{matrix} \}$$

Once we transform our input data, we implement a ε-insensitive loss function, defined in principle by the following:

$$L ( f ( f , y ) , y ) = \{ \begin{matrix}0 , & if \vert y - f ( θ , y ) \vert \leq ε \\ \vert f ( θ , y ) - 0 - ε \vert , & otherwise\end{matrix} \}$$

The result of the above loss function is that all points that fall within the error bars (grey lines in FIG. 15) are not penalized by the model, while all points outside of the error bars are penalized. In the case of SVR, we impose additional constraints to restrict the magnitude of our parameter vector, w, and minimize the error, ξ, outside of the error bars.

$${min}_{w} [ \frac{1}{2} {‖ w ‖}^{2} + C {\sum }_{i = 1}^{n} ( {ξ}_{i} + {ξ}_{i}^{*} ) ]$$

$$s . t . \{ \begin{matrix}{y}_{i} - f ( θ , y ) < ε + {ξ}_{i}^{*} \\ f ( θ , y ) - {y}_{i} \leq ε + {ξ}_{i} \\ {ξ}_{i} , {ξ}_{i}^{*} > 0 for i = 1 … n\end{matrix} \}$$

The disclosure of each of the following references is incorporated herein by reference in its entirety.

### REFERENCES

[1] E. A. Ochroch et al., “The impact of continuous pulse oximetry monitoring on intensive care unit admissions from a postsurgical care floor.,” Anesthesia and Analgesia, vol. 102, no. 3, pp. 868-875, March 2006, doi: 10.1213/01.ane.0000195583.76486.c4.
[2] G. Zonios, J. Bykowski, and N. Kollias, “Skin Melanin, Hemoglobin, and Light Scattering Properties can be Quantitatively Assessed In Vivo Using Diffuse Reflectance Spectroscopy,” Journal of Investigative Dermatology, vol. 117, no. 6, pp. 1452-1457 December 2001, doi: 10.1046/j.0022-202x.2001.01577.x.
[3] R. Kortum and E. Sevick, “Quantitative Optical Spectroscopy for Tissue Diagnosis,” Annual review of physical chemistry, vol. 47, pp. 555-606, February 1996, doi: 10.1146/annurev.physchem.47.1.555.
[4] M. W. Sjoding, R. P. Dickson, T. J. Iwashyna, S. E. Gay, and T. S. Valley, “Racial Bias in Pulse Oximetry Measurement.,” The New England Journal of Medicine, vol. 383, no. 25, pp. 2477-2478 December 2020, doi: 10.1056/NEJMc2029240.
[5] P. E. Bickler, J. R. Feiner, and J. W. Severinghaus, “Effects of skin pigmentation on pulse oximeter accuracy at low saturation.,” Anesthesiology, vol. 102, no. 4, pp. 715-719, April 2005, doi: 10.1097/00000542-200504000-00004.
[6] O. Dyer, “Pulse oximetry may underestimate hypoxaemia in black patients, study finds,” British Medical Journal, vol. 371, 2020, doi: 10.1136/bmj.m4926.
[7] A. M. Luks and E. R. Swenson. “Pulse Oximetry for Monitoring Patients with COVID-19 at Home. Potential Pitfalls and Practical Guidance.” Annals of the American Thoracic Society, vol. 17, no. 9, pp. 1040-146 September 2020, doi: 10.1513/AnnalsATS.202005-418FR.
[8] W. Liu and H. F. Zhang, “Photoacoustic imaging of the eye: A mini review.,” Photoacoustics, vol. 4, no. 3, pp. 112-123, September 2016, doi: 10.1016/j.pacs.2016.05.001.
[9] Hu, D.-N. (2017 November 16). Methodology for evaluation of melanin content and Production of Pigment Cells in Vitro. Wiley Online Library. Retrieved Mar. 10, 2022, from https://onlinelibrary.wiley.com/doi/10.1111/j.1751-1097.2007.00228.x
[10] Enoch, A. J., English, M., & Shepperd, S. (2016). Does pulse oximeter use impact health outcomes? A systematic review. Archives of disease in childhood, 101 (8), 694-700. https://doi.org/10.1136/archdischild-2015-309638
[11] Chan, E. D., Chan, M. M., & Chan, M. M. (2013 March 13). Pulse oximetry: Understanding its basic principles facilitates appreciation of its limitations. Science March Direct. Retrieved 9, 2022, from https://www.sciencedirect.com/science/article/pii/S095461111300053X#bib3
[12] Nitzan, M., Romem. A., & Koppel, R. (2014). Pulse oximetry: fundamentals and technology update. Medical devices (Auckland, N.Z.), 7, 231-239. https://doi.org/10.2147/MDER.S47319
[13] Castro, D., Patil, S. M., & Keenaghan, M. (2021 September 20). Arterial blood gas. StatPearls. Retrieved Mar. 9, 2022, from https://www.ncbi.nlm.nih.gov/books/NBK536919/
[14] Guyton A C, Hall J E. Textbook of Medical Physiology. 9th ed. Philadelphia: WB Saunders; 1996.
[15] Wagner P D. Ventilation, blood flow and gas exchange. In: Mason R J, Broaddus V C, Murray J F, Nadel J A, editors. Murray and Nadel's Textbook of Respiratory Medicine. Philadelphia: WB Saunders; 2005.
[16] Castro D, Patil S M, Keenaghan M. Arterial Blood Gas. [Updated 2021 Sep. 20]. In: StatPearls [Internet]. Treasure Island (FL): StatPearls Publishing; 2021 January-. Available from: https://www.ncbi.nlm.nih.gov/books/NBK536919/
[17] Cellular respiration. Cellular Respiration—an overview|ScienceDirect Topics. (n.d.). Retrieved Mar. 9, 2022, from https://www.sciencedirect.com/topics/neuroscience/cellular-respiration
[18] Mannheimer, P. D. (2007 December). The light-tissue interaction of pulse oximetry. LWWV. Retrieved Mar. 9, 2022, from https://journals.lww.com/anesthesia-analgesia/fulltext/2007/12001/the_light_tissue_interaction_of_pulse_oximetr y.3.aspx?casa_token=Jb834bcmFx4AAAAA % 3ASTy372agZsYk7xHLI8xvzA x69tCpuvLKspKTcUBe9DFiM1Wlw95IBSquc0tPHL2PIKgn8mz0EsLs-WIKp6Omuh4
[19] FDA. (n.d.). Pulse oximeter accuracy and limitations: FDA Safety Communication. U.S. Food and Drug Administration. Retrieved Mar. 9, 2022, from https://www.fda.gov/medical-devices/safety-communications/pulse-oximeter-accuracy-and-limitations-fda-safety-communication?utm_source=STAT % 2BNewsletters&utm_campaign=46659 efca1-MR_COPY_14&utm_medium=email&utm_term=0_8cab1d7961-46659efca1-150709213 #interpretation
[20] De Leeuw, S. M., Smit, N. P. M., Van Veldhoven, M., Pennings, E. M., Pavel, S., Simons, J. W. I. M., & Schothorst, A. A. (2001). Melanin content of cultured human melanocytes and UV-induced cytotoxicity. Journal of Photochemistry and Photobiology B: Biology, 61 (3), 106-113. https://doi.org/10.1016/s1011-1344 (01) 00168-3
[21] Karsten, A. E., & Smit, J. E. (2012). Modeling and verification of melanin concentration on human skin type. Photochemistry and photobiology, 88 (2), 469-474. https://doi.org/10.1111/j. 1751-1097.2011.01044.x
[22] Amaro-Ortiz, A., Yan, B., & D'Orazio, J. A. (2014). Ultraviolet radiation, aging and the skin: prevention of damage by topical cAMP manipulation. Molecules (Basel, Switzerland), 19 (5), 6202-6219. https://doi.org/10.3390/molecules19056202
[23] Briley-Saebo, K., & Bjornerud, A. (2000 August). Accurate de-oxygenation of ex vivo whole blood using Sodium Dithionite. Retrieved Mar. 9, 2022, from https://cds.ismrm.org/ismrm-2000/PDF7/2025.PDF
[24] White, D. C., & Teasdale, P. R. (1966). The oxygenation of blood by hydrogen peroxide: in vitro studies. British journal of anaesthesia, 38 (5), 339-344. https://doi.org/10.1093/bja/38.5.339
[25] Austin M A, Wills K E, Blizzard L et al. Effect of high flow oxygen on mortality in chronic obstructive pulmonary disease patients in prehospital setting: randomised controlled trial. BMJ 2010; 341: c5462.
[26] Castillo A, Sola A, Baquero H et al. Pulse oxygen saturation levels and arterial oxygen tension values in newborns receiving oxygen therapy in the neonatal intensive care unit: is 85% to 93% an acceptable range? Pediatrics 2008; 121:882-9.
[27] Ewer A K, Middleton L J, Furmston A T et al. Pulse oximetry screening for congenital heart defects in newborn

It will be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. A pulse oximeter comprising:
a first sensor for producing a first measurement indicative of a melanin concentration in a portion of the skin of a subject;
a second sensor for producing a second measurement indicative of light absorbed by melanin and arterial blood of the subject; and
a blood oxygen saturation percentage measurement generator for generating a third measurement based on the first and second measurements, wherein the third measurement is indicative of a percentage of oxygen saturation of arterial blood of the subject.

2. The pulse oximeter of claim 1 wherein the first sensor comprises at least one light source and at least one light detector configured to measure light absorbed by the melanin in the skin of the subject.

3. The pulse oximeter of claim 2 wherein the at least one light source comprises at least one light emitting diode (LED) for emitting light in a wavelength range and the at least one first light detector comprises at least one photodiode configured to measure light of the wavelength range emitted by the at least one LED.

4. The pulse oximeter of claim 3 wherein the wavelength range of the light emitted by the at least one LED includes an ultraviolet to violet wavelength range.

5. The pulse oximeter of claim 4 wherein the ultraviolet to violet wavelength range comprises about 390 nanometers (nm) to about 410 nm.

6. The pulse oximeter of claim 1 wherein the second sensor comprises at least one light source and at least one light detector configured to measure light absorbed by the arterial blood of the subject.

7. The pulse oximeter of claim 6 wherein the at least one light source comprises at least one first light emitting diode (LED) for emitting light in a wavelength range and the at least one first light detector comprises at least one photodiode configured to measure light of the wavelength range emitted by the at least one LED.

8. The pulse oximeter of claim 7 wherein the wavelength range emitted by the at least one LED includes at least one wavelength selected from a red wavelength range and at least one wavelength selected from an infrared wavelength range.

9. The pulse oximeter of claim 8 wherein the at least one wavelength selected from the red wavelength range comprises 660 nanometers (nm) and the at least one wavelength selected from the infrared wavelength range comprises 940 nm.

10. The pulse oximeter of claim 1 wherein the blood oxygen saturation percentage measurement generator is configured to utilize outputs from the first and second sensors to estimate the melanin concentration.

11. The pulse oximeter of claim 1 wherein the blood oxygen saturation percentage measurement generator is configured to calculate an adjusted modulation ratio based on the first and second measurements and use the adjusted modulation ratio to generate the third measurement indicative of the percentage of oxygen saturation of the arterial blood of the subject.

12. The pulse oximeter of claim 1 comprising signal enhancing optics for enhancing signals detected by the first and second sensors.

13. The pulse oximeter of claim 12 wherein the signal enhancing optics include light blockers, optical filters, or polarizers.

14. The pulse oximeter of claim 1 comprising post-acquisition signal enhancers for enhancing signals produced by the first and second sensors.

15. The pulse oximeter of claim 14 wherein the post-acquisition signal enhancers implement wavelet transforms or machine learning algorithms to filter or smooth signals detected by the first and second sensors.

16. A method for melanin-concentration-adjusted pulse oximetry, the method comprising:
detecting, using a first sensor, a first measurement indicative of a melanin concentration in a portion of the skin of a subject;
detecting, using a second sensor, a second measurement indicative of light absorbed by melanin and arterial blood of the subject; and
generating, based on the first and second measurements, a third measurement indicative of a percentage of oxygen saturation of arterial blood of the subject.

17. The method of claim 16 wherein the first sensor comprises a melanin concentration sensor, the second sensor comprises an arterial blood light absorption sensor, and generating the third measurement includes generating an adjusted modulation ratio based on the first and second measurements.

18. The method of claim 17 wherein generating the third measurement includes mapping the adjusted modulation ratio to a blood oxygen saturation percentage value.

19. The method of claim 17 wherein the first sensor includes at least one ultraviolet-violet light source and the second sensor includes at least one red and at least one infrared light source.

20. A non-transitory computer readable medium having stored thereon executable instructions that when executed by a processor of a computer control the computer to perform steps comprising:
obtaining a first measurement generated by a first sensor and indicative of a melanin concentration in a portion of the skin of a subject;
obtaining a second measurement generated by a second sensor and indicative of light absorbed by melanin and arterial blood of the subject, and
generating, based on the first and second measurements, a third measurement indicative of a percentage of oxygen saturation of arterial blood of the subject.
